(19) United States
(12) Patent Application Publication
(10) Pub. No.: US 2008/0140062 A1
(43) Pub. Date: Jun. 12, 2008

(54) Microwave Applicator

(76) Inventor: Nigel Cronin, Lane Bath (GB)

Correspondence Address: Cesari and McKenna, 88 Black Falcon Avenue, Boston, MA, 02210, US

(21) Appl. No.: 11/632,324

(22) PCT Filed: Jul. 15, 2005
(86) PCT No.: PCT/GB05/02776

§ 371 Date: Sep. 17, 2007

(30) Foreign Application Priority Data
Jul. 16, 2004 (GB) 0415973.7

(51) Int. Cl. A61B 18/18

(52) U.S. Cl. 606/33

(57) ABSTRACT

A microwave applicator (2) which comprises an antenna (10) for transmitting microwaves. Electrically conductive pins (24) such as metallic pins are present near the antenna in order to create regions (26) of low or substantially null magnetic field in the magnetic field surrounding the antenna. A sensor such as a thermocouple can be placed in one of the regions, and the effects of the magnetic field on the sensor are reduced or substantially eliminated.

### TECHNICAL FIELD

This invention relates to a microwave applicator, and in particular to the use of sensors in such an applicator.

### BACKGROUND TO THE INVENTION

International Patent application No. WO95/04385 discloses apparatus for the treatment of menorrhagia which involves applying microwave electromagnetic energy at a frequency which will be substantially completely absorbed by the endometrium, monitoring the temperature to ensure that the endometrium tissue is heated to about 60°, and maintaining the microwave energy for a period of time sufficient to destroy the cells of the endometrium. A temperature sensor, in the form of a thermocouple, is used to monitor the temperature on an ongoing basis during the treatment.

If the thermocouple is constructed of metal, the magnetic field created by the microwaves around the device induces currents and/or direct heating of the thermocouple, which leads to errors in the temperature reading. As a result of this problem, it has been the practice to take temperature readings either when the power is off, which precludes real-time measurement, or using non-metallic sensors, such as fibre-optic sensors, which are much more expensive.

### SUMMARY OF THE INVENTION

According to the invention, a microwave applicator comprises an applicator head adapted to transmit microwaves, and is characterised by further comprising at least one cancellation element positioned in the magnetic field of the microwaves so as to support induce currents which generate corresponding magnetic cancellation fields to create at least one region with a minimum magnetic field for placement of a sensor therein.

Thus, the microwave applicator can be used with a sensor such as a thermocouple positioned in said region of minimum magnetic field so as to reduce or eliminate the unwanted effects of magnetically induced currents in the sensor.

Preferably, the applicator head incorporates an antenna that transmits the microwaves, and each cancellation element is positioned alongside the antenna. Preferably, the antenna and cancellation element are embedded within a body of dielectric material.

Preferably, the cancellation element is arranged such that the region of minimum magnetic field is positioned close to an external surface of the body of dielectric material.

Preferably, the applicator is powered via a coaxial cable, and the antenna is an extension of the inner conductor of the coaxial cable into the body of dielectric material.

Preferably, the cancellation element is an elongated element which is arranged parallel to the antenna and is shorter in length than the antenna. Preferably, the cancellation element comprises a metallic conductor such as a metallic pin.

Preferably, a sensor such as a temperature sensor is located in the region of minimum magnetic field.

Advantageously, two or more cancellation elements are present within the body of dielectric material. Each element produces a region of minimum magnetic field in the magnetic field surrounding the microwave applicator. Thus multiple sensors may be placed at different locations around the applicator, each sensor being positioned within one of the regions of minimum magnetic field.

### BRIEF DESCRIPTION OF THE DRAWINGS

The invention will now be described, by way of example only, with reference to the accompanying drawings, in which:

FIG. 1 shows a cross-section of an embodiment of a microwave applicator according to the invention;

FIG. 2 shows a rear-end view of the applicator of FIG. 1;

FIG. 3 shows a front-end view of the applicator of FIG. 1;

FIG. 4 shows a graph of the electromagnetic field surrounding the applicator of FIG. 1 when in use; and

FIG. 5 shows the embodiment of FIG. 1 with component dimensions added.

### DETAILED DESCRIPTION OF EMBODIMENTS OF THE INVENTION

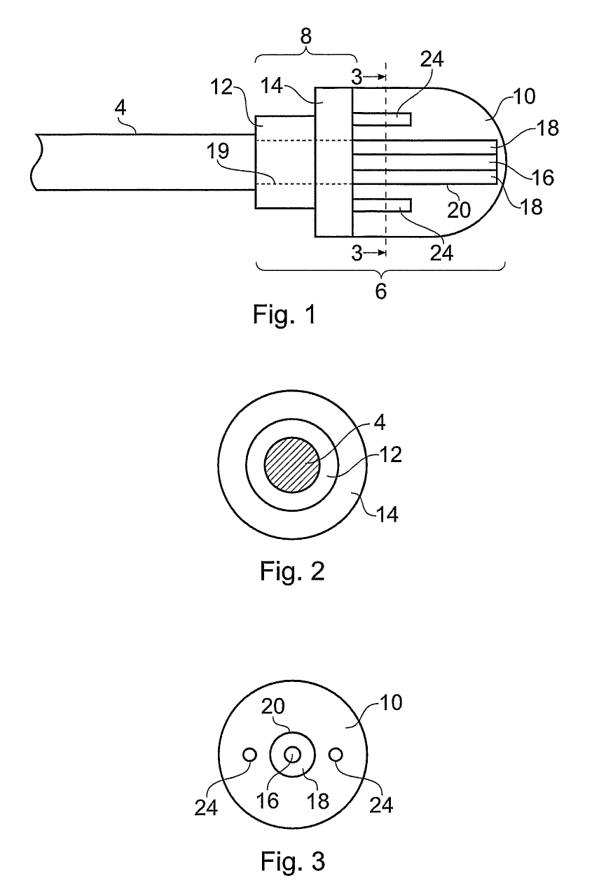

The microwave applicator 2 shown in FIG. 1 comprises a coaxial cable 4 and an applicator head 6 fastened to one end 7 of the coaxial cable 4. Only a length of the cable 4 is shown for clarity.

The coaxial cable 4 comprises inner and outer concentric conductors 16, 15 with an electrically insulating dielectric material 18 therebetween and with an outer insulating cover.

The applicator head 6 comprises a base 8, to which a body of dielectric material 10 is attached. The base 8 comprises a disc-shaped base wall 14 and a coaxial sleeve 12. The sleeve 12 receives the end 7 of the coaxial cable 4. The radius of the base wall 14 is greater than that of the sleeve 12. The body of dielectric material 10 is attached directly to the face of the base wall 14 opposite the sleeve 12 and projects co-axially from it.

The inner conductor 16 and the electrically insulating dielectric material 18 of the coaxial cable 4 extend beyond the end of the outer conductor 15, through a central aperture 19 in wall 14 and into the body of dielectric material 10. The inner conductor 16 thus forms an antenna 20 within the body of dielectric material 10.

The body of dielectric material 10 presents a smooth interface between antenna 20 and the surrounding body tissue. The dielectric constant of the body of dielectric material 10 is chosen such that a maximum amount of the microwaves propagates into surrounding body tissue under treatment, and internal reflections within the body of dielectric material 10 are minimised. A dielectric constant value of 25 is preferred for this purpose.

Two metallic pins 24 are also embedded within the body of dielectric material 10. They are positioned around the antenna 20 diametrically opposite each other. The pins 24 extend from the base wall 14 into the body of dielectric material 10 parallel to the antenna 20, and are shorter in length than the antenna. FIG. 3 shows a cross-section of the microwave applicator 2 along a plane 3-3 shown in FIG. 1, and shows the positions of the pins 24 more clearly.

The end of the coaxial cable 4 remote from the applicator head 6 is connected to a microwave power supply (not shown). When power is applied to the coaxial cable 4, microwaves are transmitted by the antenna 10. These microwaves have associated with them a magnetic field. This magnetic field induces currents in each pin 24, and these induced currents, in turn, produce a magnetic field. The induced magnetic field modifies the magnetic field associated with the microwaves, creating a region outwardly of each pin 24 where the magnetic field strength is substantially null.

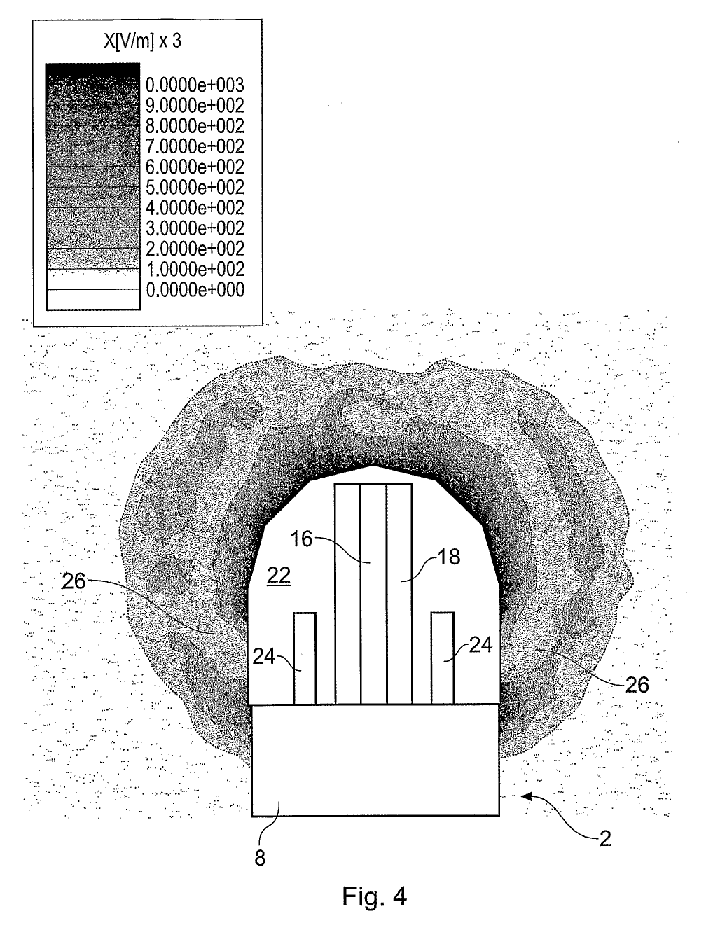

FIG. 4 shows a graph of the electromagnetic field produced by a computer model of the microwave applicator device 2 when microwaves are being transmitted. Darker regions indicate a stronger electromagnetic field. The graph shows two regions 26 of substantially null electromagnetic field radially outwards of the pins 24. These null regions 26 would not be present without the pins 24.

The pins 24 are sized and positioned so that the regions 26 of substantially null electromagnetic field are close to the surface of the body of dielectric material 10.

In use, a temperature sensor can be fixed to the outside surface of the body of dielectric material 10 within one of the regions 26. Thus, the electromagnetic field surrounding the device does not substantially affect readings taken by such a sensor.

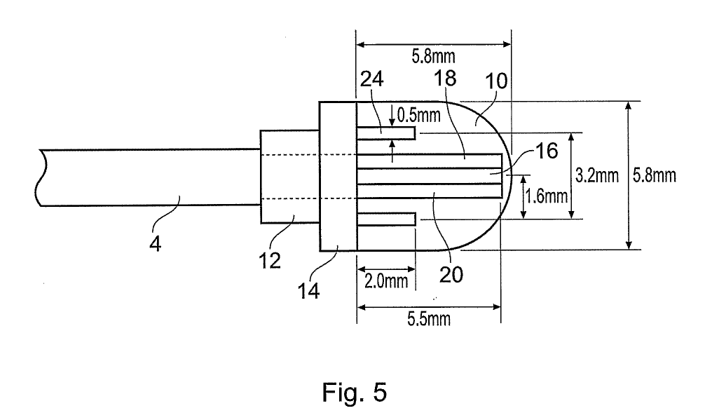

FIG. 5 shows typical dimensions in millimetres of the components, including the pins 24, which create the regions 26 at the positions shown in FIG. 4.

Typically microwave applicator 2 operates at a frequency around 9.2 Ghz and at a power of 30 w, although different frequencies and/or power ratings may be used depending on the application.

In alternative embodiment of the invention there may be just one pin, or two or more, each producing a respective null region for a sensor.

The pins 24 in the above described embodiment are metallic, however the invention is not limited to metallic pins. The pins 24 may be of any material having a sufficient electrical conductivity to influence the magnetic field surrounding the applicator head 6 and to reduce the magnetic field in the regions where it is intended to place a sensor. The pins 24 must also be electrically isolated, having no galvanic connections to other components, only the inductive connection with the electromagnetic field.

1. A microwave applicator comprising:
a microwave antenna having an axis, the microwave antenna adapted to transmit microwaves uniformly about the axis, and to generate a first magnetic field; and
at least one electrically conductive element proximate to the microwave antenna, the at least one electrically conductive element configured such that the first magnetic field induces a current in the at least one electrically conductive element thereby producing a second magnetic field about the at least one electrically conductive element, wherein the at least one electrically conductive element is further configured such that the second magnetic field modifies the first magnetic field so as to create a null region, while leaving the uniform transmission of microwaves outward of the at least one electrically conductive element predominantly intact.

2. The microwave applicator of claim 1 further comprising a dielectric material surrounding at least a portion of the microwave antenna.

3. The microwave applicator of claim 2 wherein the at least one electrically conductive element is disposed within the dielectric material.

4. The microwave applicator of claim 3 wherein the dielectric material has an outer surface, the microwave applicator further comprising a temperature sensor disposed on the outer surface in the null region produced by the at least one electrically conductive element.

5. The microwave applicator of claim 4 further comprising a disc-shaped base having a first face that is perpendicular to the axis of the microwave antenna, the disc-shaped based being spaced from an end of the microwave antenna, wherein the dielectric material is attached to the first face of the disc shaped base and extends therefrom.

6. The microwave applicator of claim 5 wherein the at least one electrically conductive element includes two elongate pins, each pin extending from the first face of the disc-shaped base into the dielectric material parallel to the axis of the microwave antenna.

7. The microwave applicator of claim 3 wherein the dielectric material has a dielectric constant of about 25.

8. The microwave applicator of claim 3 wherein the at least one electrically conductive element is a pin.

9. The microwave applicator of claim 8 wherein the pin is parallel to the axis of the microwave antenna.

10. A microwave applicator comprising:
a microwave antenna having an axis, the microwave antenna adapted to generate an electromagnetic field of microwaves extending radially about the axis for a full circumference;
at least one electrically conductive element disposed within the electromagnetic field, the at least one electrically conductive element configured to produce a zone of reduced electromagnetic energy having a narrow circumference; and
a sensor positioned at the zone of reduced electromagnetic energy.

11. The microwave applicator of claim 10 wherein the sensor is a temperature sensor.

12. The microwave applicator of claim 11 further comprising a dielectric material surrounding at least a portion of the microwave antenna.

13. The microwave applicator of claim 12 wherein the at least one electrically conductive element is disposed within the dielectric material.

14. The microwave applicator of claim 13 wherein the dielectric material has a dielectric constant of about 25.

15. The microwave applicator of claim 13 wherein the at least one electrically conductive element is a pair of metal pins disposed on either side of the microwave antenna.

16. A microwave applicator comprising:
a microwave antenna having an axis, the microwave antenna adapted to generate a first electromagnetic field of microwaves, the first electromagnetic field extending circumferentially about the axis; and
at least one electrically conductive element disposed within the first electromagnetic field, the at least one electrically conductive in response to being disposed in the first electromagnetic field, generating a second electromagnetic field of microwaves, wherein
the first and second electromagnetic fields interact to produce a null region adjacent to the microwave applicator, the null region having a narrow circumference.

17. The microwave applicator of claim 16 further comprising a dielectric material surrounding at least a portion of the microwave antenna.

18. The microwave applicator of claim 17 wherein the at least one electrically conductive element is disposed within the dielectric material.

19. The microwave applicator of claim 18 further comprising a temperature sensor positioned at the null region.

20. The microwave applicator of claim 19 wherein the at least one electrically conductive element is a metal pin extending parallel to the axis of the microwave antenna.
